(19) United States
(12) Patent Application Publication
(10) Pub. No.: US 2021/0186779 A1
(43) Pub. Date: Jun. 24, 2021

(54) CAST REMOVING TOOL

(71) Applicant: Chung-Sing Lin, Tainan (TW)

(72) Inventor: Chung-Sing Lin, Tainan (TW)

(73) Assignee: Chung-Sing LIN, Tainan (TW)

(21) Appl. No.: 16/724,389

(22) Filed: Dec. 23, 2019

(51) Int. Cl. A61F 15/02; B23D 45/16

(52) U.S. Cl. CPC A61F 15/02; B23D 45/16

(57) ABSTRACT

A cast removing tool includes a handle having a battery received therein, and a switch is connected to the handle and electrically connected to the battery. A link is connected to the handle and a motor is connected to the link. The motor has an output shaft which protrudes beyond the link and is connected to a cutting tool. The cutting tool is activated by the motor. A protection member is a curved member and connected to the handle. The protection member extends to be located beneath the cutting tool. The protection member is inserted between the cast and the patient's limb so as to avoid the cutting tool to hurt the patient.

### BACKGROUND OF THE INVENTION

### 1. Fields of the Invention

The present invention relates to a cast removing tool, and more particularly, to a cast removing tool with a cutting member to remove cast from patient's limb while protecting the patient from being hurt by the cutting member.

### 2. Descriptions of Related Art

When a bone injury happens such as a bone fracture, a cast is applied to the injury area to hold the bone once the bone is set so that the bone does not move. The cast has to be removed when the bone fracture problem is fixed. The conventional way to remove the cast is to carefully use a saw to cut the cast and then remove the cast from the patient's limb. The cutting process should be taken carefully to avoid hurting the patient.

The present invention is intended to provide a cast removing tool including a protection member to eliminate the drawbacks mentioned above.

### SUMMARY OF THE INVENTION

The present invention relates to a cast removing tool and comprises a handle having a battery received therein. A switch is connected to the outside of the handle and electrically connected to the battery. A link is connected to the front end of the handle and a motor is connected to the link. The motor has an output shaft which protrudes beyond the link. A cable is electrically connected between the motor and the battery. A cutting tool is connected to the output shaft of the motor so as to be driven by the motor. A protection member is connected to the handle and is a curved member. The protection member extends to be located beneath the cutting tool.

The advantage of the present invention is that the protection member is located between the cutting tool and the patient limb so that the cutting tool cannot touch the patient's limb.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the cast removing tool of the present invention;

FIG. 2 is a side view of the cast removing tool of the present invention;

FIG. 3 is a side view to illustrate the use of the cast removing tool of the present invention, and

FIG. 4 is a perspective view to illustrate the use of the cast removing tool of the present invention.

### DETAILED DESCRIPTION OF THE INVENTION

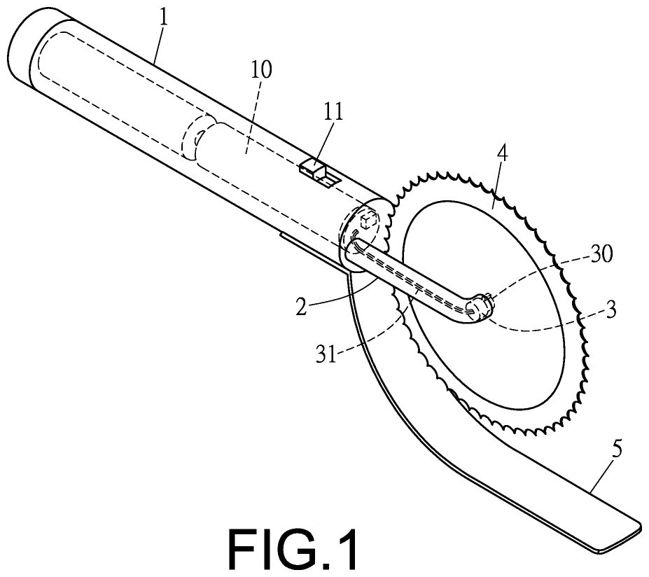

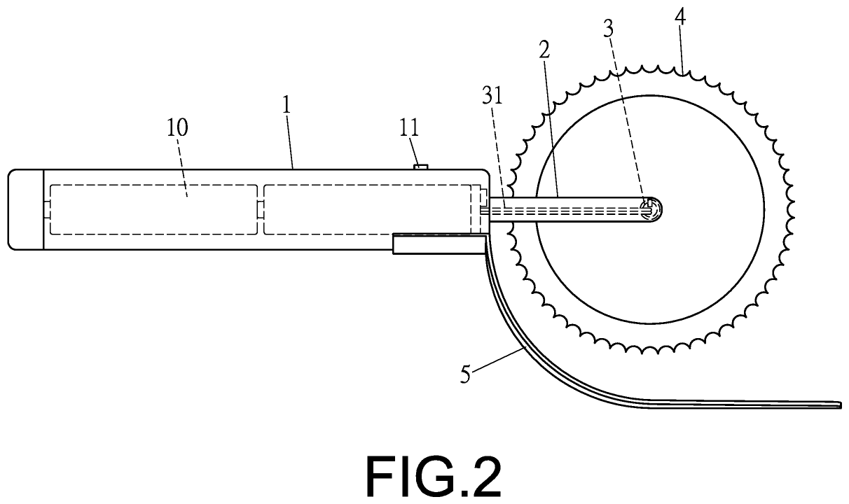

Referring to FIGS. 1 and 2, the cast removing tool of the present invention comprises a handle 1, a cutting tool 4 and a protection member 5. The handle 1 has a battery 10 received therein. A switch 11 is connected to the outside of the handle 1 and electrically connected to the battery 10. A link 2 is connected to the front end of the handle 1 and a motor 3 is connected to a bent portion of the link 2. The motor 3 has an output shaft 30 which protrudes beyond the bent portion of the link 2. A cable 31 is electrically connected between the motor 3 and the battery 10. The cutting tool 4 is connected to the output shaft 30 of the motor 3. In this embodiment, the cutting tool 4 is a disk saw. The protection member 5 is connected to the bottom of the handle 1 and is a curved member. The protection member 5 extends to be located beneath the cutting tool 4.

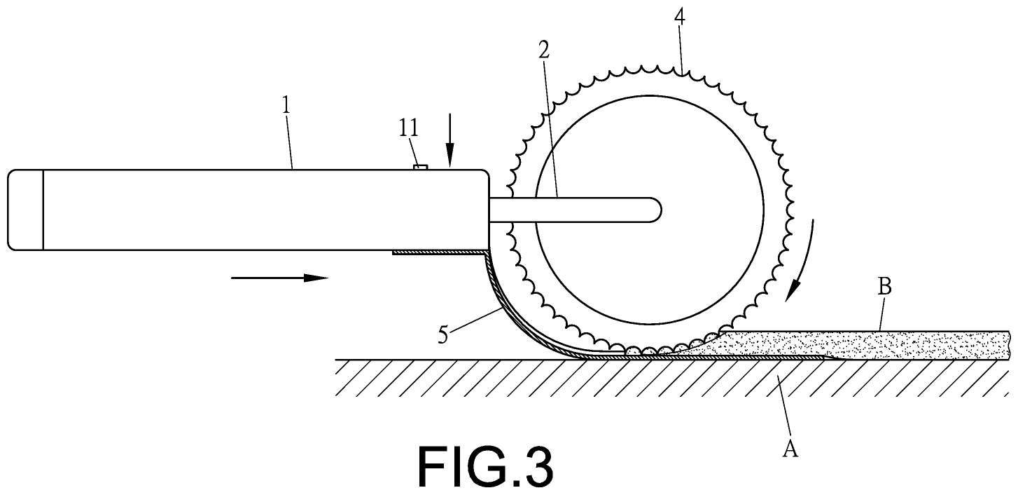

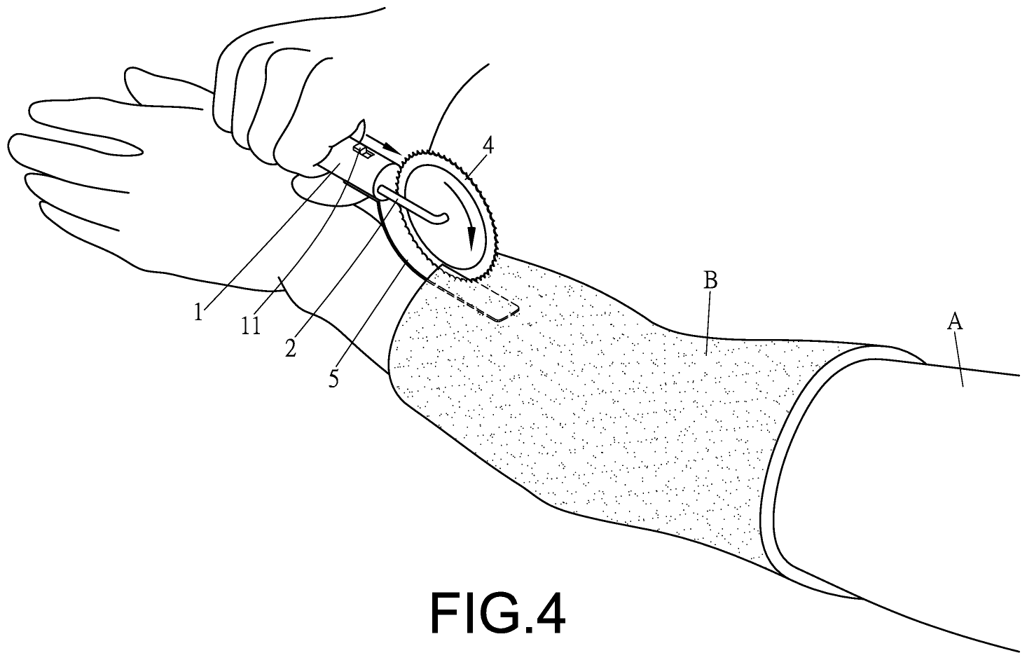

As shown in FIGS. 3 and 4, when in use, the protection member 5 is inserted between the patient's limn “A” and the cast “B”, and the cutting tool 4 is activated by operating the switch 11 on the handle 1. The handle 1 is pushed downward and the cutting tool 4 starts to cut the cast “B”. Because the protection member 5 is located between the patient's limn “A” and the cast “B”, so that the cutting tool 4 does not hurt the patient. The present invention has a simple structure and is easily to use. The patient is well protected by the protection member 5.

While we have shown and described the embodiment in accordance with the present invention, it should be clear to those skilled in the art that further embodiments may be made without departing from the scope of the present invention.

What is claimed is:

1. A cast removing tool comprising:
a handle having a battery received therein, a switch connected to an outside of the handle and electrically connected to the battery;
a link connected to a front end of the handle and a motor connected to the link, the motor having an output shaft which protrudes beyond the link, a cable being electrically connected between the motor and the battery;
a cutting tool connected to the output shaft of the motor, and
a protection member connected to the handle and being a curved member, the protection member extending from the handle and located beneath the cutting tool.
